(19) United States
(12) Patent Application Publication
(10) Pub. No.: US 2006/0052825 A1
(43) Pub. Date: Mar. 9, 2006

(54) Surgical implant alloy

(76) Inventors: Mark H. Ransick, West Chester, OH (US); Michael A. Murray, Bellevue, KY (US); Darrel M. Powell, Cincinnati, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/263,437

(22) Filed: Oct. 31, 2005

Related U.S. Application Data
(63) Continuation-in-part of application No. 10/462,553, filed on Jun. 16, 2003.

(51) Int. Cl. A61B 17/08

(52) U.S. Cl. 606/219

(57) ABSTRACT

The present invention provides a surgical implant which can be made of a metal that corrodes while implanted in the tissue of a patient. The surgical implant can include a electrical insulator for reducing the conductivity of the implant. The surgical implant can be in the form of a staple, and insulator can be in the form of an anodized surface layer.

### RELATED APPLICATION

This application cross-references, incorporates by reference, and claims priority to U.S. patent application Ser. No. 10/462,553 “Surgical Implant with Preferential Corrosion Zone”, filed Jun. 16, 2003 and published as US2004/0254608.

### FIELD OF THE INVENTION

The present invention relates, in general, to the field of surgery and, more particularly, to surgical implants including a metallic portion.

### BACKGROUND

Surgeons implant a wide variety of metallic, ceramic, and polymeric materials into patients. Surgeons use metallic implants primarily for orthopedic purposes, but additional applications include wound closure (internal and external), reconstructive surgery, cosmetic surgery, wire leads, heart valve parts, aneurysm clips, and dental uses. Because metals have favorable mechanical properties, including elasticity, deformability, and stability, metallic implants are generally less bulky than their non-metallic counterparts - - - an important precondition for application to minimally invasive surgery. Metallic implants must withstand and function within the body environment at least for a certain period of time. Therefore, the rate and type of structural degradation, via corrosion and other processes while in vivo, is an important consideration in the design of surgical implants. In addition, corrosion of metallic implants is an important consideration for biocompatibility, due to the release of metal ions into the body environment.

Some of the metals currently used for surgical implants include stainless steel (AISI type 316L), cobalt-chromium-molybdenum-carbon, cobalt-chromium-tungsten-nickel, cobalt-nickel-chromium-molybdenum, titanium, Ti-6Al-4V, Ti-3Al-2.5V, and tantalum. These metals transition from an active to a passive state by developing a protective surface oxide film when used as implants and are highly corrosion resistant in saline environments such as in the body.

The body recognizes surgical implants as foreign objects, potentially leading to local and possibly systemic reactions. Permanent metallic implants are particularly undesirable for young patients because retention for decades is unavoidable. Some metallic implants including, for example, surgical staples, clips, and vascular stents, may be constructed of metals that corrode quickly in the body. The corrosion by-products are harmlessly absorbed by the body or passed through the digestive system. For example, a surgical staple made from commercially pure iron may corrode in animal soft tissue within a few weeks, but the staple would have sufficient structural integrity for a long enough period of time, usually several days, to allow healing of the tissues involved. The surgical staple may also be made of other absorbable metals, including carbon steel. The absorption of small amounts of corrosion by-products (for iron or carbon steel, the primary by-product is iron oxide or rust,) is not known to have any significant, deleterious effect on the body. The ferromagnetic property of iron and carbon steel is a factor relative to their compatibility with NRI (magnetic resonance imaging), although the very small mass of some implants, such as surgical staples, and the very short time they are present in the body before corroding and being absorbed, allows the beneficial use of such materials. Other benefits of absorbable staples include reducing scatter on X-ray images, minimizing future adhesions, and avoiding staple lines in future surgical procedures.

Corrosion resistance of a metal is specific to a number of factors, including composition, changes in metallurgical heat treatment, microstructural phases present, and surface finish. The rate of corrosion of a metal can be slowed or halted by applying a coating, such as a moisture barrier, that shields the metal from the corrosive environment. Conversely, creating an even harsher corrosive environment can accelerate the corrosion rate of a metal. In addition, it is possible to cause the corrosion process to be focused on a localized area of the metal. By using these principles and biasing the corrosion process to take place at a desired rate and/or at a desired location of the metal, it is possible to design a metallic, surgical implant that corrodes within the body in a beneficial manner.

Each of the many surgical implants that may be made from an absorbable metal has a shape that is designed specifically for its deployment into tissue and its initial, primary function, such as holding tissue layers together during wound healing. As the implant corrodes, the ability of the implant to perform its primary function degrades. Biasing the corrosion rate and location on the implant allows the implant to fragment in a desirable way during the early stages of the corrosion process. For example, physical attributes of the implant important for deployment into tissue are not necessarily desirable thereafter while implanted in the body. The sharp tips of a surgical staple are necessary for penetration into tissue during deployment, but can cause prolonged pain or irritation to the patient thereafter. Procedures with such post-surgical complaints by patients include inguinal hernia repair and hysterectomy (in which a male sexual partner experiences the discomfort.) Also, in some situations, it would be advantageous for the implant to corrode in a specific manner, so that the ability of the implant to perform its primary function even improves. For example, surgical staplers commonly referred to in the art as circular staplers are used to perform an end-to-end or end-to-side anastomosis of hollow organs such as the large or small intestines. The surgeon uses the circular stapler to deploy a plurality of tiny, surgical staples evenly spaced apart in a pair of concentric circular staplelines (or more simply, “staple circles”) around a lumen, in order to connect the two organs together in fluid communication. Each staple is formed into a “B-shape” to clinch tissue layers together. A ring of relatively inelastic scar tissue forms over these staple circles. By using surgical staples that initially corrode and fragment from “B-shapes” into “two half B-shapes”, the primary tissue holding function of the staples is not compromised, yet the staple circles are more flexible and easily dilated.

Surgical implants formed of magnesium alloys are known in the art. Some surgeons may use electrocautery or other electro-surgical devices near an implant, such as a staple, in order to stop any residual bleeding from areas near the implant. When the surgeon uses a monopolar electrocautery pencil, the surgeon places the patient on a grounding pad and may touch the pencil to one implant. When there is a series of implants, such as a line of staples applied by a commercial surgical stapler, it is desirable that the current does not “spark” from one implant to the next. It is desirable that the electrical current takes a path directly to the grounding pad. One disadvantage of using a staple formed of magnesium is that “sparking” can occur if an electro-surgical device is used in close proximity to a staple line formed from magnesium staples.

### SUMMARY OF THE INVENTION

Applicants have recognized the desirability of providing a surgical implant that reduces the likelihood of sparking or otherwise providing a electrical conduction path, and in particular, in providing a surgical implant comprising magnesium with a reduced likelihood of forming a conduction path for electricity.

Applicants have also recognized the desirability of providing a surgical implant that includes an alkaline earth metal, such as magnesium, in combination with another metal that promotes corrosion. Suitable materials for promoting corrosion include, without limitation, iron, copper, cobalt, nickel, and combinations thereof.

In one embodiment, the present invention provides a surgical implant, such as a surgical staple or clip, having a conductive portion, and where at least a portion of the conductive portion is covered with an electrical insulator. The insulator can be employed to reduce or minimize sparking or electrical activity when an RF device or other electro-surgical device is used near a staple line.

The insulator can be an applied coating or film, such as a parylene film, a bioabsorbable coating or film (such as an absorbable synthetic polymer), or a non-metallic film. Alternatively, the insulator can be a surface layer, such as an oxidation layer that is less conductive than the underlying conductive portion.

In one embodiment, the insulator comprises an oxidation layer formed on the surface of a surgical staple formed from a metallic alloy. The surgical staple can be formed of a magnesium alloy, and the insulator can comprise an anodized oxidation layer formed on the surface of the magnesium alloy.

The oxidation layer can have a thickness of at least about 0.00005 inch. In one embodiment, the layer can be between about 0.00005 inch and about 0.0015 inch, more particularly between about 0.00005 inch and about 0.0001 inch.

The present invention also provides a surgical implant that is formed of an alloy including at least one component, such as a metallic element, for promoting corrosion. In one embodiment, the surgical implant is employed to corrode within the body in less than about 200 days, and the implant can be formed of an alloy of an alkaline earth metal (such as magnesium) and at least one element for promoting corrosion of the implant. For example, the alloy can be a magnesium alloy with iron, cobalt, copper, or nickel in sufficient quantity to promote corrosion.

### BRIEF DESCRIPTION OF DRAWINGS

FIG. 1 is a top view of a surgical staple.

FIG. 2 is a front view of the surgical staple in FIG. 1.

FIG. 3 is a cross sectional view taken at line 3-3 of FIG. 2 having an electrical insulator layer 11 with thickness T.

### DETAILED DESCRIPTION OF THE INVENTION

All percentages are by weight unless otherwise indicated.

The present invention provides a surgical implant. In one embodiment, the present invention is a surgical implant comprising an electrical insulator. While the surgical implant disclosed in the drawings is in the form of a surgical staple, it will be understood that the surgical implant of the present invention can take on various other forms, including without limitation the form of a surgical clip, stent, or bone anchor.

For instance, surgeons use metallic implants for orthopedic purposes, but additional applications include wound closure (internal and external) reconstructive surgery, cosmetic surgery, wire leads, heart valve parts, aneurysm clips, and dental uses. Because metals have favorable mechanical properties, including elasticity, deformability, and stability, metallic implants are generally less bulky than their non-metallic counterparts, which can be important for application to minimally invasive surgery. Metallic implants withstand and function within the body environment at least for a certain period of time.

Some metals used for surgical implants corrode more quickly than others. These metals can provide implants that are absorbed by the body after a period of time so that the patient does not carry an implant after the implant is no longer needed. Among the metals that corrode relatively quickly and are absorbed by tissues are certain metals such as magnesium.

Some surgeons may use electrocautery near an implant to stop any residual bleeding from areas near the implant. When the surgeon uses a monopolar electrocautery pencil, the surgeon places the patient on a grounding pad and may touch the pencil to one implant. When there is a series of implants, such as a line of staples applied by a commercial surgical stapler, it is desirable that the current does not “spark” from one implant to the next. It is desirable that the electrical current takes a path directly to the grounding pad.

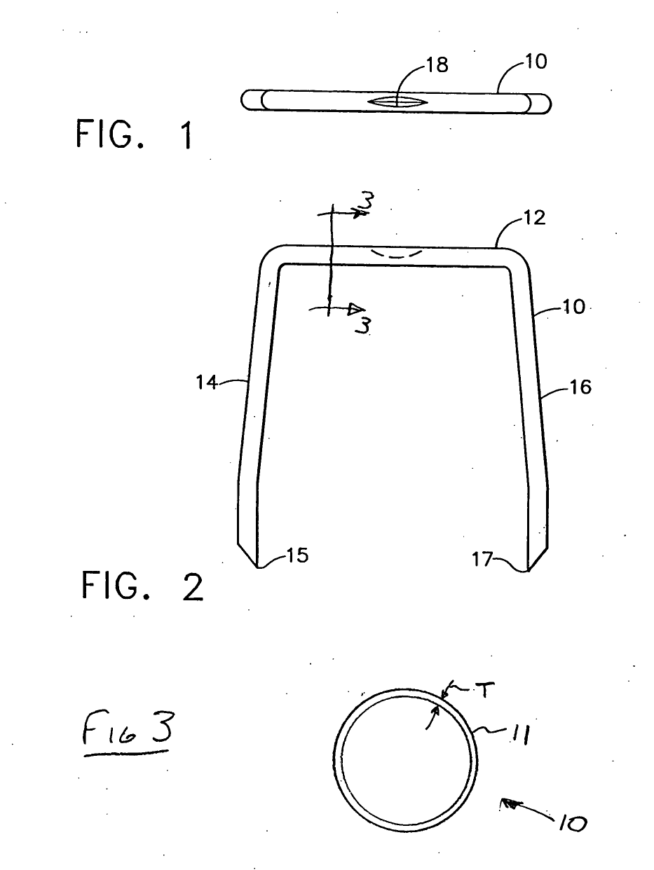

An suitable staple is illustrated in FIGS. 1 and 2. FIG. 1 shows a top view and FIG. 2 shows a front view of a staple 10. By way of non-limiting example, Staple 10 can be made from 0.279 mm (0.011 inch) diameter wire and comprises a first leg 14, a second leg 16, connected by a crown 12. First leg 14 and second leg 16 can each be approximately 5.51 mm (0.217 inches) long. Crown 12 can be approximately 3.96 mm (0.156 inches) wide. First leg 14 can have a first tip 15 and second leg 16 has a second tip 17. In the embodiment in FIGS. 1-2, an indentation 18 can be provided which is located approximately in the middle of crown 12. Indentation 18 can be employed to provide a preferential corrosion zone, as set forth in above referenced US patent application “Surgical Implant with Preferential Corrosion Zone”, incorporated herein by reference.

It has been found that implants made from magnesium alloys implanted into tissue will exhibit sparking when electrocautery is applied to one implant in the series. A visible spark will be seen to travel from one implant to the next. The visible sparking can make a surgeon uncomfortable with the performance of the staples and the staple line. However, it is still desirable to use magnesium because of the absorbability and the corrosion properties that it offers.

Without being limited by theory, it is believed that the sparking occurs with magnesium because of its high electrical conductivity. Magnesium has a conductivity of about 225 (milliohm-cm)⁻¹ (225/(milliohm-cm) ). By comparison, Titanium conductivity measures about 24 (milliohm-cm)⁻¹. Because of magnesium's high electrical conductivity, the impedance of a series of implants of magnesium alloy can be lower than a path directly from one implant to the grounding pad. Therefore, electrical current may travel along the implant line instead of by the desired path through the tissue surrounding the implant.

In one embodiment, the present invention provides a relatively highly conductive implant with an electrical insulator, so that the implant is less electrically conductive than it would be otherwise, thereby reducing or otherwise retarding sparking. In embodiment, the lower conductivity can be achieved by forming an insulating layer on the surface of the implant, such as thin oxidation layer. In one embodiment, the electrical insulating layer can be formed by anodizing the surface of an implant formed of a magnesium alloy, or alternatively, by applying a substance to the surface that has lower conductivity than magnesium, such as non-conductive film or coating. In one embodiment, a film of parylene having a thickness of between about 2 microns to about 50 microns can be employed. In another embodiment, a coating or film formed of an absorbable synthetic polymer and a medicant such as an antibiotic, hemostatic, or pain relief composition. Suitable absorbable materials include, without limitation, polyglycolic acid, polylactide, polylactic acid, polylactide coglycolide, and poly caprolactone. One suitable polymer is that employed in commercially available Vicryl® brand polyglactin 910 products.

FIG. 3 is a cross-sectional view of the staple 10 of FIGS. 1 and 2. FIG. 3 illustrates a surface layer 11 having a thickness T. The surface layer 11 can be formed by creating a relatively thin layer of oxidation on the surface of a magnesium alloy implant. For instance, the surface layer 11 can be formed by anodizing the magnesium alloy. One suitable magnesium alloy is a magnesium alloy comprising aluminum, zinc, and iron.

Some or all of the staple 10 can be insulated by the surface layer 11. Generally, at least about 50 percent of the surface of an implant would be covered, and more particularly, at least about 80 percent of the surface could be covered by the insulator layer 11. If desired, substantially the entire surface of the staple 10 can be covered, either before or after the tips are formed. If desired, the layer 11 can be applied or formed selectively so as to provide a preferential corrosion layer.

The layer 11 can have a thickness of at least about 0.00005 inch. In one embodiment, the thickness T can be between about 0.00005 inch and about 0.0015 inch, more particularly between about 0.00005 inch and 0.0005 inch, and still more particularly between about 0.00005 inch and about 0.0001 inch.

Suitable alloys for use in forming a surgical implant having a surface layer 11 for providing an electrical insulator include, but are not limited to, AZ31 and Az91 magnesium alloys. A surgical implant formed from a magnesium alloy can be anodized to form a surface layer 11 by using a micro arc oxidation technique, as set forth in U.S. Pat. No. 4,978,432, incorporated by reference in its entirety herein.

By way of non-limiting prophetic example, a staple 10 with layer 11 can be made from commercially available magnesium alloy AZ31 wire stock, having about 50 parts per million iron. Prior to forming the wire into the form of a staple, the wire can be anodized with the MAGOXID-COAT® process available from Luke Engineering and Manufacturing Company, Wadsworth, Ohio. A process utilizing non-chromate micro arc oxidation can be used to provide a surface layer 11 having a thickness of about 0.0005 inches.

Staples 10 formed in such a manner can be used in a mechanical surgical stapler, or in a stapler specifically designed to use the magnesium staples produced in this example. The staples could be deployed to anastomose tissue in either an open or an endoscopic procedure. The procedure could be, for example, a bowel anastomosis following removal of a portion of the bowel for cancer surgery, an anastomosis of a portion of the small intestine to the stomach or another portion of the bowel as a part of a gastric bypass operation for weight reduction, or a closing of the vaginal cuff as a portion of a hysterectomy. “Surgical Stapling Technique for Radical Hysterectomy”, Fanning et al., Gynecologic Oncology 55, 179-184 (1994) discloses the use of surgical staples in radical hysterectomy, and is incorporated herein by reference.

Surgical implant fasteners having the surface layer 11 may be used in various surgical procedures and with various surgical devices. For instance, such implants can be use to approximate the rectus fascia for operative procedures, such as to repair ventral hernias. Fasteners such as those described in U.S. Pat. No. 6,706,048, the entire contents of which are hereby incorporated herein by reference, can be provided with a surface layer 11 according to the present invention. Such fasteners could then be used in a procedure in which the surgeon incises the medial border of the rectus fascia of a patient and locates a jaw of an applicator tool into the envelope formed by the rectus sheath that surrounds the abdominus rectus muscles. The surgeon locates a second jaw within the second rectus sheath. The jaws of the tool can be advanced to the location where a first fastener can be placed after using the jaws to pull the sheaths together. An applicator tool described in the '048 patent can also have a releasable hinge mechanism, such as a removable pin, to allow the jaw members of the applicator tool to separate completely to be placed separately within the rectus sheaths and then to be linked together at the hinge mechanism. In one embodiment, a plurality of fasteners made of a magnesium alloy and having a surface layer 11 according to the present invention may be used along the length of a jaw of the applicator tool, so that the fasteners degrade at an advantageous rate and have an electrically insulative layer 11.

Staples of various configurations, including without limitation those used in a circular stapler, can be provided with a surface layer 11. U.S. Pat. No. 5,309,927 is incorporated herein by reference in its entirety, including for its teaching with respect to the use of a circular stapler for performing anastomosis.

Surgical implants having a surface layer 11 according to the present invention can also be used in performing bypass procedures, such as in the digestive tract. US Patent Application 2004/0087977 is incorporated herein by reference in its entirety, including but not limited for its teaching regarding laparascopic techniques for bypass procedures.

The protective layer 11 can also be employed with ligating clips and other ligating surgical devices. The following US patents/applications are incorporated by reference in their entirety, including but not limited for their disclosure related to surgical clips: U.S. Pat. No. 4,799,481; U.S. Pat. No. 5,163,945; U.S. Pat. No. 5,340,360; U.S. Pat. No. 5,431,668; Re 35,525; US 2003/0225423; US 2004/0116948; and US 2005/0090838.

In one embodiment, the present invention provides a surgical implant, such as a staple 10, formed of a metallic alloy having at least one constituent for accelerating corrosion. Past investigators have recommended alloy materials to reduce the rate of corrosion of an implant material. However, for absorbable implants, it may be desirable to increase the rate of corrosion of a material, because some surgical implants are implanted into areas that receive little or no blood flow. These implants have been found to last longer in the body than is needed for the adjoining tissues to heal properly. Accordingly, in one embodiment of the present invention, it may be desirable to increase the rate of corrosion, either separately or in combination with providing an insulator layer 11.

Without being limited by theory, increasing iron content of a magnesium alloy can cause the implant to corrode or degrade more quickly. By way of example, increasing the amount of iron in an AZ31 magnesium alloy can decrease the time required for corrosion in a salt-spray test. By way of further example, increasing the amount of iron in an AZ91 magnesium alloy will also decrease the time to corrode a test sample in a salt-spray test.

For instance, and without being limited by theory, it is believed that an AZ31 magnesium alloy with about 50 parts per million iron will promote relatively rapid corrosion as compared to a pure iron staple. More rapid corrosion can be advantageous in areas of the body with little oxygen supply.

In one embodiment, the magnesium alloy can comprise between about 1 percent and about 7 percent aluminum, about 0.5 percent and about 1.5 percent zinc, and at least about 50 parts per million iron. Without being limited by theory, it is believed that the presence of iron in sufficient quantity can promote corrosion of the staple 10 in a desired time period.

In another embodiment, a suitable magnesium alloy comprises between about 1 percent and about 7 percent aluminum, about 0.5 percent and about 1.5 percent zinc, and between about 50 parts per million and about 300 parts per million iron.

In yet another embodiment, the magnesium alloy comprises between about 1 percent and about 5 percent aluminum, about 0.5 percent and about 1.5 percent zinc, and between about 50 parts per million and about 200 parts per million iron.

In still another embodiment, the magnesium alloy comprises between about 2.5 percent and about 3.5 percent aluminum, about 0.5 percent and about 1.5 percent zinc, and between about 100 parts per million and about 175 parts per million iron.

While the above embodiments include iron for promoting corrosion, it is also possible to include other elements, such as nickel, copper, and/or cobalt to promote corrosion.

While numerous alternate embodiments of the present invention, it will be obvious to those skilled in the art that such embodiments are only examples, and that there are numerous variations and substitutions possible without departing from the invention. It will also be understood that various features and element of the claimed invention can be alternatively described in terms of a means for performing the function provided by the feature and/or element. We intend that the invention be limited only by the spirit and scope of the appended claims.

What is claimed:

1. A surgical implant, the implant comprising a magnesium alloy comprising between about 1 percent and about 7 percent aluminum; about 0.5 percent and about 1.5 percent zinc, and at least about 50 parts per million iron.

2. The implant of claim 1 wherein the implant comprises a magnesium alloy comprising between about 1 percent and about 5 percent aluminum; about 0.5 percent and about 1.5 percent zinc, and between about 50 parts per million iron and about 500 parts per million iron.

3. The implant of claim 1 wherein the implant comprises a magnesium alloy comprising between about 1 percent and about 5 percent aluminum; about 0.5 percent and about 1.5 percent zinc, and between about 50 parts per million iron and about 200 parts per million iron.

4. The implant of claim 1 wherein the implant comprises a magnesium alloy comprising about 2.5 percent aluminum and about 3.5 percent aluminum; about 0.5 and about 1.5 percent zinc; and between about 100 parts per million iron and about 175 parts per million iron.

5. A surgical implant, the implant comprising an alloy of an alkaline earth metal and a metal for promoting corrosion of the implant within about 200 days.

6. A surgical implant wherein the implant comprises an alloy of an alkaline earth metal, aluminum, zinc, and a metal for promoting corrosion of the implant, wherein the metal for promoting corrosion is selected from the group consisting of: iron, nickel, copper, and cobalt.

7. A method for fastening tissue comprising the steps of:
providing a surgical fastener comprising a magnesium alloy comprising between about 1 percent and about 7 percent aluminum; about 0.5 percent and about 1.5 percent zinc, and at least about 50 parts per million iron;
providing a device for applying the surgical fastener to tissue; and
applying the surgical fastener to tissue.

8. The method of claim 7 wherein the surgical fastener comprises a staple.

9. The method of claim 7 wherein the method comprises anastomosis of two organs.

10. The method of claim 7 wherein the method comprises: transecting hollow organ tissue; inserting an anastomotic device transluminally through the transected hollow organ tissue; approximating the transected hollow organ tissue; and fixing the hollow organ tissue with at least one of the surgical fasteners.

11. The method of claim 7 wherein the method comprises approximating the rectus fascia.

12. The method of claim 7 wherein the method comprises closing an open end of a vaginal cuff.
